(19) United States
(12) Patent Application Publication
(10) Pub. No.: US 2025/0319225 A1
(43) Pub. Date: Oct. 16, 2025

(54) DEVICE AND METHOD FOR DISPENSING AND/OR DIFFUSING VOLATILE SUBSTANCES, ESPECIALLY FOR DISPENSING AND/OR DIFFUSING FRAGRANCES AND/OR ACTIVE SUBSTANCES IN AIR CARE AND/OR PEST CONTROL

(71) Applicant: CTR, LDA, Samora Correia (PT)

(72) Inventor: Harold AUGIER, Zhongshan City (CN)

(21) Appl. No.: 18/868,871

(22) PCT Filed: May 25, 2022
(86) PCT No.: PCT/EP2022/064175

§ 371 Date: Nov. 25, 2024

(51) Int. Cl. A61L 9/03; A01M 1/20

(52) U.S. Cl. CPC A61L 9/037; A01M 1/2072; A01M 1/2077; A61L 9/032; A61L 2209/11; A61L 2209/134

(57) ABSTRACT

A device for dispensing and/or diffusing volatile substances has a storage tank for a substance to be released into the environment, an electric heating device, and a delivery engine coupled to the heating device. The delivery engine has a substance-receiving area which is in fluid communication with the substance received in the storage tank. The substance is conveyable from the substance-receiving area to a substance-discharging area of the delivery engine, at which the substance is discharged into an air stream to generate a substance air stream enriched with the substance. A controller actuates the electric heating device and/or an air flow generation device during operation of the device. The electric heating device forms a structural unit in the form of a pod heater and has at least the storage tank, the electric heating element and the delivery engine.

The invention relates to a device for dispensing and/or diffusing volatile substances, in particular for dispensing and/or diffusing fragrances and/or active substances in air care and/or pest control, according to the preamble of claim 1, and a method for operating a device for dispensing volatile substances, in particular for dispensing fragrances and/or active substances in air care and/or pest control, according to the preamble of claim 18.

Devices for dispensing volatile substances are well known and usually comprise a container in which a substance to be dispensed is contained. For example, the container contains a capillary wick which protrudes above the container with a free end of the wick and which is in contact with the substance to be dispensed in such a way that the substance is conveyed to the free end of the wick by the capillary action of the wick. An electrical heating element is regularly assigned to the free end of the wick, by means of which heat can be applied to the free end of the wick in order to be able to release the substance accumulating in the free end of the wick into the environment or evaporate it even faster. Such a structure is known from WO 98/58692 A1, for example. In order to be able to adjust the degree of evaporation and thus the evaporation capacity, said WO 98/58692 A1 also provides for the container and wick to be stored in the housing of the device in such a height-adjustable manner that the relative position of the wick to the heating device can be changed.

With such a design, the evaporation rate and thus the release rate of the substance to be released can be influenced over a longer period of time. However, there is the problem that the air flow generated and enriched with the substance to be dispensed, which escapes into the environment via an outlet opening in the housing wall, cannot flow out of the housing fast enough and is influenced, for example, by air turbulence arising within the housing, so that undesirable deposits and condensation of the substance to be dispensed on the inner walls of the housing occur to a certain extent. Furthermore, with such devices, especially if they are arranged close to a wall, there is a risk that the outflowing substance air flow will change into the desired unstable convection state just above the device. In a relatively low room, e.g. a device mounted on a wall near a socket, this often leads to an insufficiently effective distribution of the substance to be emitted in the room or to undesired deposits of the substance to be emitted on the wall.

Accordingly, it is an object of the present invention to create a device and a method for the dispensing and diffusing, in particular for the evaporation of volatile substances, by means of which an effective outflow of the substance air stream can be ensured or a sufficient distribution of the substance to be delivered in the room can be achieved.

This object is solved with the features of the independent patent claims. Advantageous embodiments are subject of the dependent claims.

The invention relates to a device for dispensing and/or diffusing volatile substances, e.g. liquid or gel substances, in particular for dispensing and/or diffusing fragrances and/or active substances. The device may be used basically anywhere, but the use and application of the device in the field of air care and pest control is particularly preferred.

The device comprises at least one storage tank containing a substance to be released into the environment.

The device also comprises an electrical heating device having at least one electrical heating element by means of which heat is generated, preferably for heating and/or evaporating the substance to be emitted.

Furthermore the device comprises at least one delivery engine, preferably a capillary element, being coupled to the electrical heating device, preferably being coupled to the at least one electrical heating element of the electrical heating device, in a direct or indirect heat-transferring manner, with the delivery engine having at least one substance-receiving area which is in fluid communication with the substance received in the storage tank, so that the substance is conveyable from the at least one substance-receiving area, preferably by means of capillary action, to at least one substance-discharging area of the delivery engine, at which the substance is discharged into at least one air stream, preferably into at least one pure or clean air stream, and at least one substance air stream enriched with substance is generated.

The device also comprises at least one air flow generating device by means of which the at least one air stream to be enriched with substance, preferably the at least one pure or clean air stream to be enriched with substance, is generated.

The device further comprises at least one control device by means of which the actuation of the electrical heating device and/or of the air flow generating device is controlled during operation of the device, preferably at intervals. It is of particular advantage if the at least one control device controls both the actuation of the at least one air flow generating device and the actuation of the electrical heating device during operation of the device, preferably at intervals.

According to the invention the electrical heating device forms a structural unit in the form of a pod heater and comprises at least the at least one storage tank, the at least one electrical heating element, preferably formed by a heating wire or a heating coil, and the at least one delivery engine.

With such a device, a targeted, fast-flowing substance air stream can be generated, which is not only effectively and quickly enriched with substance, but also released into the environment at a high flow rate. The relatively high flow velocity makes it possible to effectively distribute the substance to be released even in a larger room volume. This means that the high flow velocity allows good diffusion or dispersion of the substance to be released even in larger rooms. This depends, as already mentioned, on the one hand, on the flow velocity of the substance air stream generated, but also on the evaporator temperature set by means of the electrical heating device. As the tests carried out by the inventor have shown, the forced flow generated by the air flow generating device, in conjunction with a high substance release rate caused by the vaporisation, causes a particularly effective and rapid filling of the room with the substance or a sufficiently good distribution of the substance in the room. Due to the forced flow by means of the air flow generating device, it is also completely sufficient if the maximum temperature of the heating element, preferably a heating coil as the electrical heating element, is only between 100° C. and 280° C., particularly preferably between 100° C. and 250° C. The necessity of setting high temperatures above 300° C. is therefore no longer given.

In this context, the method according to the invention will also be discussed in detail:

Particularly preferred and advantageous is a method, in which the electrical heating device, preferably the at least one air flow generating device and the at least one electrical heating device, is controlled by means of the control device in such an interval-like manner that during each interval a substance quantity is delivered by the device which is smaller than the substance quantity temporarily stored in the delivery engine. Alternatively or additionally it can be provided that the delivery engine is enriched in the time between the individual intervals with a substance quantity which is larger than the substance quantity delivered in the next interval.

Both procedures ensure that the delivery engine is always moist or wet during 10 the heating process, so that, for example, a heating coil as an electrical heating element never acts only on a dry delivery engine, which can have a negative impact on the durability and life of both the delivery engine and the electrical heating element. For this reason it is also advantageous, in connection with the solution according to the invention, to use only low heating temperatures 15 between 100° C. and 280° C., preferably between 100° C. and 250° C.

Another positive factor in preventing overheating of the delivery engine and/or the electrical heating element is that the clean or pure air stream generated by an air flow generating device has a certain cooling function.

The inventor-side tests have further shown that the heating temperature and the speed of the air flow should preferably be adjusted so that the size of the substance particles released into the environment is 20 to 80 nm, preferably 30 to 60 nm (nm=nanometer).

A further particular advantage of the device according to the invention is that it can also be used to evaporate non-solution-based substances, such as essential oils, thus significantly reducing the amount of VOC in the air.

As the electrical heating device forms a structural unit in form of a pod heater respectively in form of a pod or podded pinheater, the electric heating element can be in direct contact with the delivery engine, which considerably improves the heat transfer between the electric heating element and the delivery engine. Alternatively or additionally at least a part of the electrical heating element can even be embedded in the delivery engine. Notwithstanding the design of the electric heating device as a pod heater, it is clear that the electric heating element and/or the capillary element and/or the storage tank may be, as a preferred option, replaceably or exchangeably mounted as parts of the pod heater.

According to a particularly preferred embodiment, the device comprises a housing that surrounds a housing interior. The housing has at least one air inlet and at least one air outlet. Furthermore, the device comprises at least one air guide channel which, starting from the at least one air inlet, is guided to the substance-discharging area of the delivery engine, at which the air stream, preferably the clean air stream, is enriched with the substance and a substance air stream enriched with substance is generated, and which, starting from the substance-discharging area, is guided to at least one air outlet, via which the substance air stream is discharged to the environment. The at least one air flow generating device is operable to draw in ambient air from outside the housing via the at least one air inlet and to convey the at least one air stream to be enriched with substance, preferably to convey the at least one clean air stream to be enriched with substance, to the substance-discharging area and further to the air outlet. The above-mentioned advantages of fast diffusion come to bear even more strongly with such a specific design of the device.

According to a preferred embodiment, the delivery engine is formed by a capillary element and/or has at least one through-flow channel whose channel wall forms the substance delivery region, with the at least one through-flow channel being also a component of the air guide channel. With such a design, a compact overall construction is created, in which the delivery engine is in direct contact with the substance to be dispensed, whereby the delivery engine conveys the substance to be dispensed (preferably taken up by means of capillary action) directly and immediately via the substance-discharging area into the through-flow channel, through which the air stream, preferably the clean air stream, passes and which builds a component of the air guide channel, so that effective substance dispensing into the pure or clean air stream is possible. This enables a fast, functionally reliable and effective delivery of a substance to an air flow for a desired delivery of a substance into a room with the greatest possible distribution effect.

The at least one, preferably exactly one, through-flow channel of the delivery engine preferably has a first opening which connects to a first section of the air guide channel, preferably in a gas-tight manner. The first section of the air guide channel being guided from an air inlet to the through-flow channel. The through-flow channel of the delivery engine further has a second opening opposite the first opening, through which the substance air stream flows out. The second opening forms the housing-side air outlet or connects to a second section of the air guide channel which is led to the housing-side air outlet and/or forms the housing-side air outlet. This connection is also preferably gas-tight. This type of construction ensures that the through-flow channel is optimally integrated into the air guide channel and thus the clean air stream arriving at the first opening is enriched with substance as it flows through the through-flow channel, before continuing to flow through the second opening as a substance-enriched air flow.

According to a further aspect of present invention, the device has or forms a reservoir area which has or forms the electrical heating device respectively the pod heater. This allows a very easy integration of the electrical heating device into a device or housing of a device. According to a preferred embodiment, the reservoir area forms the electrical heating device and may have a first end wall, a side wall and a second end wall spaced opposite the first end wall. With such design of the, for example barrel-shaped, reservoir area or electrical heating device, it is preferred that the delivery engine is supported on the first end wall, opens out with the first opening in the first end wall and extends in the interior of the electrical heating device, surrounded by the substance received therein, over a partial region of the interior in a chimney-like manner. It is particularly preferred that the second section of the air guide channel extends from the second opening of the delivery engine in the direction of the second end wall, preferably widens in a funnel shape and/or forms the housing-side air outlet. This type of construction provides a particularly compact design, which is particularly advantageous for relatively small device types without risking losses with regard to the effectiveness of the distribution of the substance to be discharged in the room. In this context, a design in which the second section of the air guide channel widens in a funnel shape from the second opening of the delivery engine towards the second end wall is also particularly preferred. The funnel-shaped widening allows a particularly preferred, unhindered but nevertheless directed release of the substance air flow into the environment.

For a compact overall design of the electrical heating device and/or the reservoir area, it is advantageous if the delivery engine is accommodated approximately centrally in the interior of the electrical heating device and/or reservoir area.

It is further advantageous that the housing has a technical area comprising at least the air flow generating device and/or the control device or at least the air flow generating device, the control device and the at least one air inlet. In this way, an advantageous spatial separation between individual components of the device can be achieved, so that individual housing areas, for example in particular in conjunction with a multi-part design of the housing for easy access or simple replacement of components, can be divided into technically useful subunits.

For effective heat transfer, the electrical heating element may be in direct contact with the delivery engine and/or at least a partial area of the electrical heating element may be embedded in the delivery engine. A design in which the delivery engine is a capillary element and/or has a hollow cylindrical shape with a central through-flow channel is also particularly preferred. It is preferably provided that the electrical heating element formed by a heating coil is wound around the flow channel with one or more turns resting on the outer circumferential surface of the delivery engine and/or is wound around the through-flow channel embedded in the hollow cylindrical wall of the delivery engine. This provides a highly effective, quickly heatable delivery engine which also has a compact design.

The electric heating element, preferably formed by a heating coil, most preferably by a heating coil with several turns or windings, is preferably coupled to a power or energy supply device which supplies the electric heating element with energy and heats it, controlled by the control device. For example, the energy supply may be a battery or accumulator. Plug or cable solutions, for example for an energy supply via a socket, are of course also possible at any time.

This electrical heating device respectively pod heater may, for example, be formed by a removable, separate component, which may be advantageous for replacement actions.

In connection with such a reservoir area, it is also advantageous if the electrical heating element of the electrical heating device is, in the assembled state, at least partially accommodated inside the housing and/or inside the reservoir area of the device, for example in a centrally located position.

According to another preferred embodiment, the device, preferably the housing of the device, further comprises a technical area comprising at least the at least one control device and/or the at least one air flow generating device, preferably comprising at least the at least one air flow generating device, the at least control device and the at least one air inlet.

In principle, the device or the housing of the device can be made from one-piece. However, a multi-piece device or housing is particularly preferred, which allows access to the interior or to certain areas. Thus, according to particularly preferred embodiment, the electrical heating device respectively pod heater and the technical area are formed by separate components which are releasably connected, preferably in such way that the electric heating device is exchangeably mounted on top of the technical area as viewed in the vertical axis direction.

The control unit or control device itself can be located at any suitable place. Particularly preferred, again for a particularly compact design, is a configuration in which the control device is part of a printed circuit board (PCB) located or accommodated in the technical area.

In principle, there are also various possibilities for the arrangement of the electrical heating device/pod heater and the technical area. For example, the electrical heating device/pod heater can be located above the technical area as viewed in the vertical axis direction, whereby the at least one housing-side air inlet can then be located or arranged on the technical area in a lower and/or lateral area, preferably in the base or floor area, relative to the vertical axis direction. In this design, the first section of the air guide channel is then guided from the housing-side air inlet upwards in the vertical axis direction to the reservoir area. It is preferably provided that the first section of the air guide channel is guided approximately centrally through the technical area and the structural units accommodated and arranged there, in particular through the air flow generating device and/or the power or energy supply device. This results in an overall particularly compact design with a targeted and effective air flow.

Since the preferred design provides that the delivery engine is in direct contact with the electric heating element, it is particularly advantageous if the delivery engine is a capillary element and made from a porous ceramic and/or a porous plastic.

The air flow generating device itself can be designed in different ways. Particularly advantageous are designs in which the air flow generating device is formed by at least one fan, for example a fan wheel. But also, an air flow generating device which is formed by at least one air pump can be used, for example in the form of a piezo-controlled air pump.

The invention is described in more detail below by means of a drawing:

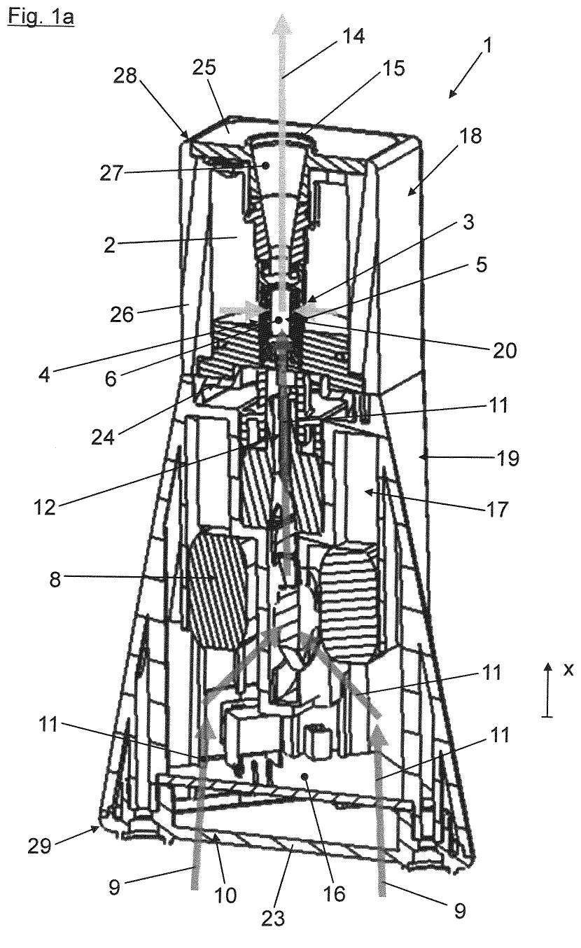

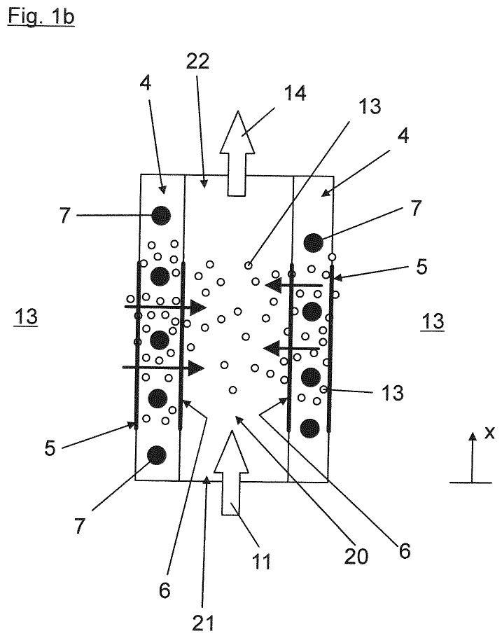

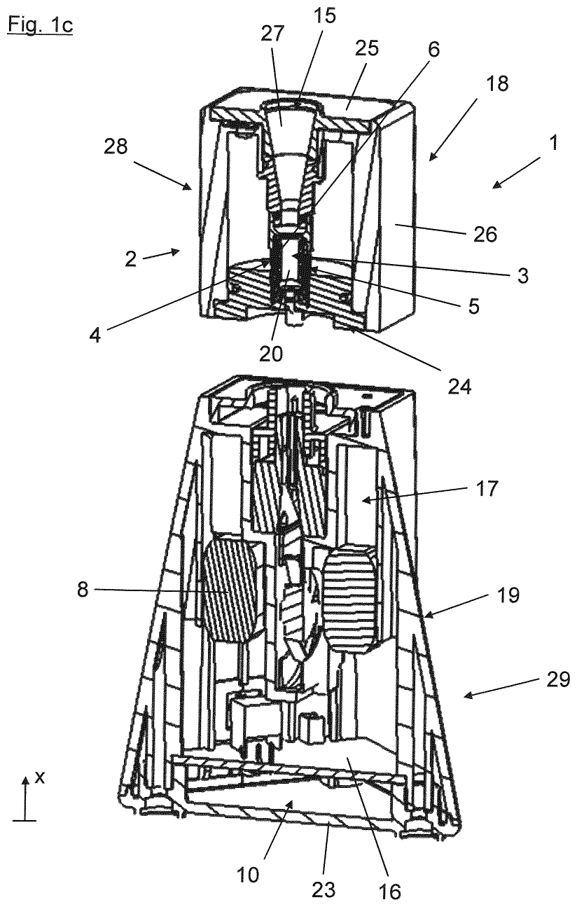

FIG. 1a a schematic, partially cut-open and perspective sketch of an exemplary type of a device for dispensing and/or diffusing volatile substances according to the invention,

FIG. 1b a schematic sketch of the operation and structure of the electric heating device of the device of FIG. 1a, which is essentially formed by a capillary element as delivery engine and a heating coil, and

FIG. 1c a schematic sketch of the design as shown in FIG. 1a, in which the reservoir unit and the technical unit form two separate and detachable components.

FIG. 1a shows, as an example and schematically, a partially cut-open, perspective representation of a device for dispensing volatile substances, for example for dispensing fragrances and/or active substances, e.g. for air care or pest control, in accordance with the invention. This device comprises a housing 1, which surrounds an interior of the housing.

Furthermore, an electric heating device 3 is part of or, as shown here, is built by a reservoir area 18.

As can be best seen in particular from the combination of FIGS. 1a and 1b, the electric heating device 3 respectively reservoir area 18 forms a structural unit in the form of a pod heater and comprises, besides the storage tank 2, a capillary element 4 as delivery engine, which is made of a porous ceramic or a porous plastic, for example. In the capillary element 4, here for example a heating coil 7 is embedded as an electrical heating element, which is coupled with a power or energy supply device 17 in a usual way not shown here in detail. The power or energy supply device 17 can, for example, have a battery or can also ensure an energy supply via an external power source.

As can be best seen in FIG. 1b, the capillary element 4, which forms part of the pod heater respectively electric heating device 3, has a substance-receiving area 5 which is in contact with the substance 13 in the storage tank 2. Furthermore, the capillary element 4 has a through-flow channel 20, the channel wall of which forms a substance discharging area 6. This through-flow channel 20 is in turn part of an air guide channel 12. Specifically, the through-flow channel 20 of the capillary element 4 has a first opening 21 (see FIG. 1b), which connects to a first section of the air guide channel 12, preferably gas-tight. This first section of the air guide channel 12 is only shown very schematically in FIGS. 1a and 1s represented by the section of the air guide channel 12, which is led from an air inlet 10 in a bottom wall 23 of the housing 1 to the through-flow duct 20. Via the air inlet 10 on the bottom wall side, which is shown here as an example, a clean air stream 11 is conveyed or sucked into the interior of the housing 1 by means of a fan 8 (as an exemplary air flow generating device) and is led in a manner yet to be described to the substance discharging area 6 of the capillary element 4, where the clean air stream 11 is enriched with the substance and a substance-enriched substance air stream 14 is generated.

To ensure that the substance air stream 14 can flow out of the flow channel 20 of the capillary element 4, this flow channel 20 has an upper or second opening 22 opposite the lower or first opening 21, here exemplarily in vertical axis direction x. Here, this second opening 22 connects to a second section of the air guide channel 12, which is led to an air outlet 15 on the housing side, whereby the connection to this second section of the air guide channel 12 is also preferably gas-tight.

As can be seen in particular in FIG. 1c, housing 1 also has a technical area 19, which here only has the fan 8, the control unit 16 and at least one air inlet 10 as examples. The reservoir area 18 (which here forms/builds the electrical heating device 3 respectively pod heater) and the technical area 19 are here formed by two separate components, which can be connected to each other detachably or removably.

The reservoir area 18 here also has, for example, a first end wall 24, a side wall 26 and a second end wall 25 opposite the first end wall 24 at a distance, whereby the capillary element 4 is supported on the first end wall 24, opens out with the first opening 21 in the first end wall 24 and extends upwards like a chimney over part of the interior of the reservoir area 18, surrounded by the substance 13 incorporated therein.

The second section of the air guide channel 12, on the other hand, extends from the second opening 22 of the capillary element 4, also inside the reservoir area 18, towards the second end wall 25, whereby this second section of the air guide channel 12 here forms a funnel-shaped widening 27 which opens into the housing side air outlet 15.

The electrical heating device 3 or pod heater is preferably mounted exchangeably on top of the technical area 19 of the housing 1.

Notwithstanding the design of the electric heating device 3 as a pod heater, it is clear that the electric heating element 7 and/or the capillary element 4 and/or the storage tank 2 may be, as a preferred option, replaceably or exchangeably mounted as parts of the pod heater.

Control unit 16 is only shown here schematically and is, for example, part of a printed circuit board (PCB) included in technical area 19.

In the embodiment shown in FIGS. 1a to 1c, the reservoir area 18 is-only by way of an example—arranged above respectively on top of the technical area 19 in the vertical axis direction x, so that the reservoir area 18 forms a head or pod section 28 and the technical area 19 a base section 29, as shown in FIG. 1c.

Of course, other embodiments of the pod or head portion 28 are also possible, for example, an embodiment (not shown) in which the head portion is surrounded by and/or received within an additional cover, for example, to create a particular design effect.

The functioning of this exemplary inventive embodiment is as follows:

When the device is actuated, the control unit 16 controls the fan 8 and the electric heating device 3 or the heating coil 7 at intervals, i.e. for a certain preset period of time. This causes the capillary element 4 to heat up and the substance temporarily stored in the capillary element 4 due to capillary action, which is diffused in via the substance-receiving area 5, evaporates via the substance-discharging area 6 into the region of the through-flow channel 20, into which the clean air stream 11 drawn in via the air inlet 10 flows.

Depending on the type and winding of the heating coil, a certain temperature can be preset for the respective substance, preferably preset in such a way that the capillary element 4 does not dry out, i.e. it is always ensured that only a substance quantity is delivered which is smaller than the substance quantity temporarily stored in the capillary element 4.

In the embodiment shown here, the first section of the air guide channel 12, starting from the air inlet 10 on the housing side, is led upwards, seen in the vertical axis direction x, to the reservoir area 18, whereby the first section of the air guide channel 12 is led approximately centrally through the technical area 19 and the structural units accommodated and arranged there, namely the fan 8 and the energy or power supply device 17. It goes without saying that, in principle, other air guide channels are also possible in a housing.

In the through-flow channel 20 of the capillary element 4, the clean air flow 11 then meets the substance discharged there via the substance-discharging area 6, as already explained, so that the clean air flow 11 is enriched with substance and the substance air flow 14 already described is formed, which flows out via the funnel-shaped extension 27 and the air outlet 15 provided here as an example.

The capillary element 4 can be made of a porous ceramic and/or a porous plastic, for example.

Instead of a fan 8 as an air flow generating device, an air pump can also be used, for example a piezo-controlled air pump, which is not shown here, however.

It is preferred that the temperature of the heating coil 7 is preferably in the range between 100° C. and 250° C. and/or that during operation of the device, preferably in intermittent operation of the device, no substance quantity is discharged which is greater than the substance quantity temporarily stored in capillary element 4. This means, conversely, that the capillary element 4 must be able to accumulate a quantity of substance in the time between the individual intervals which is greater than the quantity of substance released in the next interval.

With the device according to the invention, substance particles are preferably emitted into the environment with a size of for example 20 to 80 nm, preferably 30 to 60 nm.

List of reference signs

| | |
|---|---|
| 1 | casing |
| 2 | storage tank |
| 3 | electrical heating device/pod heater |
| 4 | capillary element |
| 5 | substance receiving area |
| 6 | substance discharging area |
| 7 | heating coil |
| 8 | fan |
| 9 | ambient air |
| 10 | air inlet |
| 11 | clean air stream |
| 12 | air guide channel |
| 13 | substance |
| 14 | substance air flow |
| 15 | air outlet |
| 16 | control device |
| 17 | energy supply device |
| 18 | reservoir area |
| 19 | technical area |
| 20 | through-flow channel |
| 21 | first or lower opening |
| 22 | second or upper opening |
| 23 | bottom wall |
| 24 | first front wall |
| 25 | second front wall |
| 26 | side wall |
| 27 | funnel-shaped extension |
| 28 | head part |
| 29 | base part |

1-20. (canceled)

21. A device for dispensing and/or diffusing volatile substances, the device comprising:
at least one storage tank containing a substance to be released into the environment;
an electric heating device having at least one electric heating element for generating heat;
at least one delivery engine coupled to said electric heating device for heat-transfer, said delivery engine having at least one substance-receiving area in fluid communication with the substance received in said at least one storage tank, enabling the substance to be conveyed from the at least one substance-receiving area to at least one substance-discharging area of said delivery engine, at which the substance is discharged into an air stream;
at least one airflow generating device for generating the air stream to be enriched with the substance; and
at least one controller configured to control an actuation of at least one of said electric heating device or said at least one airflow generating device during operation of the device;
said electric heating device being a structural unit formed as a pod heater comprising the at least one storage tank, the at least one electric heating element, and the at least one delivery engine.

22. The device according to claim 21, configured for dispensing and/or diffusing fragrances and/or active substances in air care and/or pest control, wherein:
the at least one electric heating element is configured for heating and/or evaporating the substance to be emitted;
said at least one delivery engine is a capillary element directly or indirectly coupled to said at least one electric heating element for heat transfer;
the substance is conveyed from the at least one substance-receiving area by capillary action to at least one substance-discharging area of said delivery engine and the substance is discharged into at least one pure or clean air stream to generate at least one substance air stream that is enriched with the substance;
said at least one airflow-generating device is configured to generate the at least one pure or clean air stream to be enriched with the substance; and
said at least one controller is configured to control an actuation of at least one of said electric heating device or said at least one air flow generating device during operation of the device at intervals.

23. The device according to claim 21, which further comprises a housing surrounding a housing interior, and wherein:
said housing is formed with at least one air inlet and at least one air outlet, and wherein at least one air guide channel is formed, starting from said at least one air inlet, guided to said substance-discharging area of said delivery engine, at which the air stream is enriched with the substance and a substance air stream enriched with the substance is generated, and which, starting from the substance-discharging area, is guided to at least one air outlet, via which the substance air stream is discharged to the environment; and
said at least one air flow generating device is operable to draw in ambient air from outside the housing via said at least one air inlet and to convey said at least one air stream to be enriched with the substance, to said substance-discharging area and further to said air outlet.

24. The device according to claim 23, wherein said delivery engine is formed by a capillary element and/or has at least one through-flow channel with a channel wall forming said substance delivery region, with said at least one through-flow channel being also a component of said air guide channel.

25. The device according to claim 24, wherein:
wherein said through-flow channel of said delivery engine has a first opening that adjoins a first section of said air guide channel, the first section of said air guide channel being guided to said through-flow channel starting from an air inlet;
said through-flow channel of said delivery engine has a second opening which is opposite said first opening and via which the substance air stream flows out, said second opening forming a housing-side air outlet or connecting to a second section of said air guide channel which is led to the housing-side air outlet and/or forms the housing-side air outlet.

26. The device according to claim 21, further comprising a reservoir area which has or forms said electric heating device.

27. The device according to claim 26, wherein said reservoir area forms said electric heating device and has a first end wall, a side wall and a second end wall opposite and spaced from said first end wall.

28. The device according to claim 27, wherein:
said delivery engine is supported on said first end wall, opens out with the first opening in said first end wall and extends in the interior of said electric heating device, surrounded by the substance received therein, over a partial region of the interior in a chimney-like manner; and
said second section of said air guide channel extends from said second opening of said delivery engine in a direction of said second end wall, widens in a funnel shape and/or forms the housing-side air outlet.

29. The device according to claim 27, wherein said delivery engine is accommodated approximately centrally in an interior of said electric heating device and/or reservoir area.

30. The device according to claim 21, wherein said at least one electric heating element is in direct contact with said delivery engine and/or at least a partial area of said at least one electric heating element is embedded in said delivery engine.

31. The device according to claim 30, wherein said delivery engine is a capillary element and/or said delivery engine has a hollow cylindrical shape with a central through-flow channel, with a heating coil forming said electric heating element being wound around said central through-flow channel with one or more turns resting on an outer circumferential surface of said delivery engine and/or being wound around said through-flow channel embedded in the hollow cylindrical wall of said delivery engine.

32. The device according to claim 21, wherein said electric heating element is a heating coil connected to a power supply, which supplies the electric heating element with power and heats the electric heating element under control by said controller.

33. The device according to claim 21, wherein said electric heating device (3), being the pod heater, is formed as a separate, removable component.

34. The device according to claim 33, further comprising a technical area comprising at least one of said controller, said at least one air flow generating device, or said at least one air inlet.

35. The device according to claim 34, wherein said pod heater and said technical area are formed by separate components which are releasably connected, with said electric heating device being exchangeably mounted on top of said technical area as viewed in a vertical axis direction.

36. The device according to claim 34, wherein said controller is part of a printed circuit board disposed in said technical area.

37. The device according to claim 34, wherein:
said electric heating device being the pod heater lies above said technical area as viewed in a vertical axis direction;
said at least one housing-side air inlet is arranged on said technical area in a lower and/or lateral area relative to the vertical axis direction; and
a first segment of said at least one air guide channel, starting from a housing-side air inlet, is guided upwards in the vertical axis direction to a reservoir area, with the first segment of said at least one air guide channel being guided approximately centrally through said technical area and said structural units accommodated and arranged in said technical area.

38. The device according to claim 21, wherein said delivery engine is a capillary element made from at least one of a porous ceramic or a porous plastic.

39. A method for dispensing and/or diffusing volatile substances, the method comprising:
providing a device according to claim 21;
controlling the electric heating device and the at least one air flow generating device in intervals by the controller and during each interval delivering a quantity of a substance by the device which is smaller than a quantity of the substance temporarily stored in the delivery engine;
and/or
accumulating with the delivery engine in a time between the individual intervals a quantity of the substance which is greater than a quantity of the substance delivered in a following interval.

40. The method according to claim 39, which comprises operating the heating element of the electric heating device at a maximum temperature of between 100° C. and 280° C.

41. The method according to claim 39, which comprises releasing substance particles into the environment having a particle size of between 20 nm and 80 nm.
